(19) United States
(12) Patent Application Publication
(10) Pub. No.: US 2014/0066708 A1
(43) Pub. Date: Mar. 6, 2014

(54) ENDOSCOPE CAP FOR CATHETER DEPLOYMENT

(71) Applicant: NinePoint Medical, Inc., Cambridge, MA (US)

(72) Inventor: Shekhar Nimkar, Swampscott, MA (US)

(73) Assignee: NinePoint Medical, Inc., Cambridge, MA (US)

(21) Appl. No.: 13/965,379

(22) Filed: Aug. 13, 2013

Related U.S. Application Data
(60) Provisional application No. 61/693,881, filed on Aug. 28, 2012.

(51) Int. Cl. A61B 1/018; A61B 1/07

(52) U.S. Cl. CPC A61B 1/018; A61B 1/07
USPC 600/106; 600/104

(57) ABSTRACT

A surgical system is provided that includes a sleeve having an inner surface defining a first channel that extends along a first longitudinal axis and an inner surface defining a second channel that extends along a second longitudinal axis. The second channel is spaced apart from the first channel. A first surgical instrument is configured for disposal in the first channel. The first surgical instrument includes an optical probe configured for imaging. A second surgical instrument is configured for disposal in the second channel. The second surgical instrument includes an inflatable balloon. Methods and kits are also provided.

### TECHNICAL FIELD

This disclosure relates to endoscopes, and more particularly to an endoscope cap that includes a first channel for disposal of an endoscope and a second channel for disposal of devices in connection with the endoscope, such as, for example, a balloon catheter.

### BACKGROUND

Various types of devices are used in conjunction with an endoscope in order to aid in positioning a probe of the endoscope. Some of these devices include inflatable members to expand and press against an internal cavity of a patient. The probe is then used to image a portion of an internal cavity of a patient, such as, for example, the patient's esophagus.

One type of inflatable member is a balloon catheter. In general, a balloon of the balloon catheter moves between a deflated or collapsed state and an inflated or expanded state; intermediate states are also available. In operation and use, the balloon is inserted into an internal cavity of a patient when the balloon is in a deflated state. After positioning within the patient, the balloon is inflated by injecting inflation material into the balloon via a syringe, inflation bulb, or pump, for example. Some systems utilize a pressure gauge to monitor the pressure of the balloon to prevent over pressurization of the balloon.

Endoscopes are generally used in some medical procedures as an illumination and/or imaging device to illuminate and/or image an internal cavity of a patient. In order to capture clear images of the cavity tissue, a portion of the endoscope, such as, for example, an optical probe can be positioned within a balloon of a balloon catheter. The balloon with the optical probe inserted therein can then be inserted into the cavity with the balloon in the deflated state. The balloon is then inflated to provide clear access to fix the optical probe in position relative to the cavity. However, it is often difficult to properly position the optical probe in a manner that allows the optical probe to properly illuminate and/or image the cavity. Indeed, the balloon is often large and pliable making steadying the optical probe challenging. Misalignment of the optical probe can lead to poor quality images. This disclosure describes an improvement over these prior art technologies.

### SUMMARY

In one embodiment, in accordance with the principles of the present disclosure, a surgical system is provided that includes a sleeve comprising an inner surface defining a first channel that extends along a first longitudinal axis and an inner surface defining a second channel that extends along a second longitudinal axis. The second channel is spaced apart from the first channel. A first surgical instrument is configured for disposal in the first channel. The first surgical instrument comprises an optical probe configured for illumination and/or imaging. A second surgical instrument is configured for disposal in the second channel. The second surgical instrument comprises an inflatable balloon.

In one embodiment, in accordance with the principles of the present disclosure, the surgical system includes a sleeve comprising an inner surface defining a first channel that extends along a first longitudinal axis and an inner surface defining a second channel that extends along a second longitudinal axis. The second longitudinal axis is parallel to the first longitudinal axis. The first channel has a width that is greater than that of the second channel. The widths of the first and second channels are each uniform along a length thereof. The second channel is spaced apart from the first channel by a wall. The sleeve comprises a shape memory polymer or low durometer silicone. An endoscope comprises a shaft disposed in the first channel. The shaft includes an inner surface defining a first passageway having an optical probe configured for illumination and/or imaging slidably disposed therein. A balloon catheter comprising a utility tube is disposed in the second channel. The utility tube comprises an inner surface defining a second passageway configured for disposal of an inflatable balloon. The first and second channels extend between first and second end surfaces of the sleeve. The first and second end surfaces each extend perpendicular to the first and second longitudinal axes such that a length of the first channel is equal to that of the second channel.

In one embodiment, in accordance with the principles of the present disclosure, the surgical system includes a sleeve comprising an inner surface defining a first channel that extends along a first longitudinal axis and an inner surface defining a second channel that extends along a second longitudinal axis. The second longitudinal axis is parallel to the first longitudinal axis. The first channel has a maximum width that is greater than that of the second channel. The second channel is spaced apart from the first channel by a wall. A first portion of the second channel has a cylindrical cross sectional configuration and a second portion of the second channel has a tapered configuration. The sleeve comprises a shape memory polymer or low durometer silicone. An endoscope comprises a shaft disposed in the first channel. The shaft includes an inner surface defining a first passageway having an optical probe configured for illuminating and/or imaging slidably disposed therein. A balloon catheter comprises a utility tube that is disposed in the second channel. The utility tube comprises an inner surface defining a second passageway configured for disposal of an inflatable balloon. The first and second channels extend between first and second end surfaces of the sleeve. The first end surface extends at an acute angle relative to the first and second longitudinal axes and the second end surface extends perpendicular to the first and second longitudinal axes such that a length of the first channel is less than that of the second channel.

In one embodiment, in accordance with the principles of the present disclosure, a method for catheter deployment comprises: providing the surgical system described in paragraph [0005]; inserting a utility tube of the second surgical instrument into the second channel, the utility tube comprising an inner surface defining a first passageway; inserting the balloon through the first passageway with the balloon in an unexpanded orientation; selectively positioning the balloon in an internal cavity of a patient; moving the balloon from the unexpanded orientation to an expanded orientation such that the balloon engages tissue that defines the internal cavity to fix the balloon relative to the internal cavity; inserting a shaft of the first surgical instrument into the first channel, the shaft comprising an inner surface defining a second passageway; inserting the optical probe through the second passageway and into the internal cavity; and illuminating and/or imaging the internal cavity using the optical probe.

In one embodiment, in accordance with the principles of the present disclosure, kit for an endoscope comprises: the surgical system described in paragraph [0005]; one or more additional sleeves; and one or more additional second surgical instruments, the inflatable balloons of each of the second surgical instruments having a different configuration when in an expanded orientation.

### BRIEF DESCRIPTION OF THE DRAWINGS

The present disclosure will become more readily apparent from the specific description accompanied by the following drawings, in which:

FIG. 1 is a breakaway, perspective view of a surgical system in accordance with the principles of the present disclosure;

FIG. 2 is a perspective view of components of the system shown in FIG. 1, with parts separated;

FIG. 3 is a perspective view of components of the system shown in FIG. 1;

FIG. 4 is a perspective view of components of the system shown in FIG. 1;

FIG. 5 is an end view of a component of the system shown in FIG. 1;

FIG. 6 is a side, cross sectional view of a component of the system shown in FIG. 1 taken along lines A-A;

FIG. 7 is an end view of a component of a surgical system in accordance with the principles of the present disclosure;

FIG. 8 is a side, cross sectional view of the component shown in FIG. 7 taken along lines D-D;

FIG. 9 is a breakaway, side view of components of a surgical system in accordance with the principles of the present disclosure; and

FIG. 10 is a top view of a component of the system shown in FIG. 9.

Like reference numerals indicate similar parts throughout the figures.

### DETAILED DESCRIPTION

The exemplary embodiments of the surgical system and related kits and methods of use disclosed herein are discussed in terms of an endoscope cap that provides, among other benefits, channels for simultaneous deployment of an endoscope and a balloon catheter in a manner that allows a balloon of the balloon catheter to fix the optical probe in position relative to a portion of a patient's anatomy to be illuminated and/or imaged by an optical probe of the endoscope without the balloon interfering with such illumination and/or imaging. It is understood that the present disclosure is applicable to any illumination and/or imaging system that includes an optical probe that is inserted into an internal cavity of a patient to illuminate and/or obtain images of the tissue surrounding the probe.

In one embodiment, in accordance with the principles of the present disclosure, an endoscope cap is provided that includes a first passageway configured for disposal of a shaft of an endoscope and a second passageway configured for disposal of a catheter of a balloon catheter. The surgical system is effective to pull back and deploy at least a 25 mm balloon through the endoscope cap. That is, the surgical system is configured for disposal of a catheter including a balloon having a diameter of at least 25 mm when the balloon is in an expanded orientation. It is envisioned that the surgical system of the present disclosure may be used to deploy balloon catheters that include balloons having various sizes. That is, the size of the balloon is not limited by the components of the surgical system. In one embodiment, the endoscope cap comprises a clear or transparent material. In one embodiment, the endoscope cap comprises silicone. In one embodiment, the endoscope cap has a durometer of 25 Shore A.

In one embodiment, the endoscope cap has a length of ½ inch. However, it is envisioned that the endoscope cap can have any length, depending upon the requirements of a particular application. In one embodiment, the second passageway has an inner diameter of about 4.0 mm to about 4.2 mm, to allow for deployment of a balloon that has a diameter of 25 mm when in an expanded configuration. However, it is envisioned that the inner diameter of the second passageway can be adjusted to accommodate smaller and larger balloons. In one embodiment, the endoscope cap is flexible and the first passageway has a diameter that is less than that of a shaft of the endoscope such that the endoscope cap is stretched to fit on an end of the shaft. In some embodiments, the endoscope cap is a single use device that is discarded after the first use. In some embodiments, the endoscope cap has magnetic tacking capability that enables tracking in 3D.

The present disclosure may be understood more readily by reference to the following detailed description of the disclosure taken in connection with the accompanying drawing figures, which form a part of this disclosure. It is to be understood that this disclosure is not limited to the specific devices, methods, conditions or parameters described and/or shown herein, and that the terminology used herein is for the purpose of describing particular embodiments by way of example only and is not intended to be limiting of the claimed disclosure.

Also, as used in the specification and including the appended claims, the singular forms “a,” “an,” and “the” include the plural, and reference to a particular numerical value includes at least that particular value, unless the context clearly dictates otherwise. Ranges may be expressed herein as from “about” or “approximately” one particular value and/or to “about” or “approximately” another particular value. When such a range is expressed, another embodiment includes from the one particular value and/or to the other particular value. Similarly, when values are expressed as approximations, by use of the antecedent “about,” it will be understood that the particular value forms another embodiment. It is also understood that all spatial references, such as, for example, horizontal, vertical, top, upper, lower, bottom, left and right, are for illustrative purposes only and can be varied within the scope of the disclosure. For example, the references “superior” and “inferior” are relative and used only in the context to the other, and are not necessarily “upper” and “lower”.

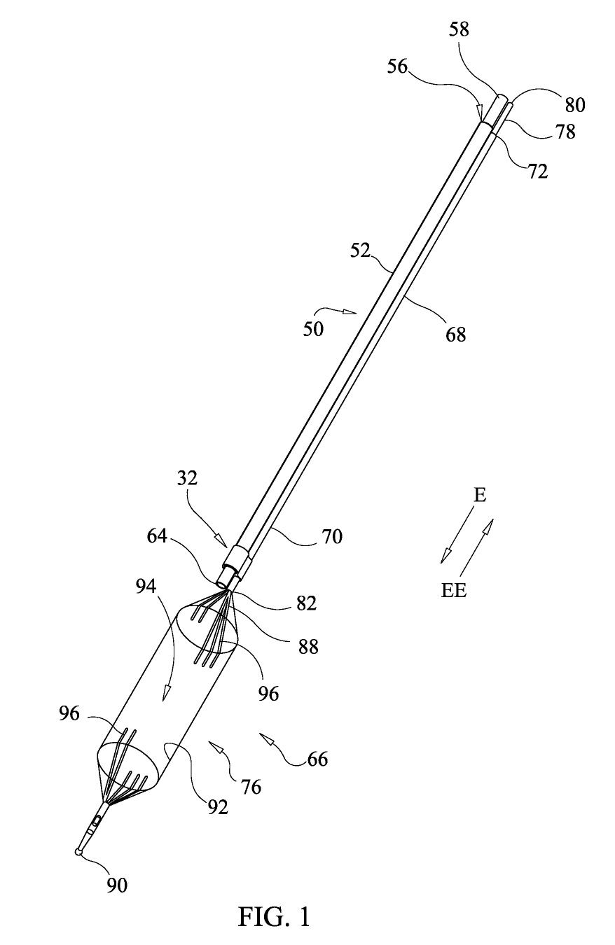

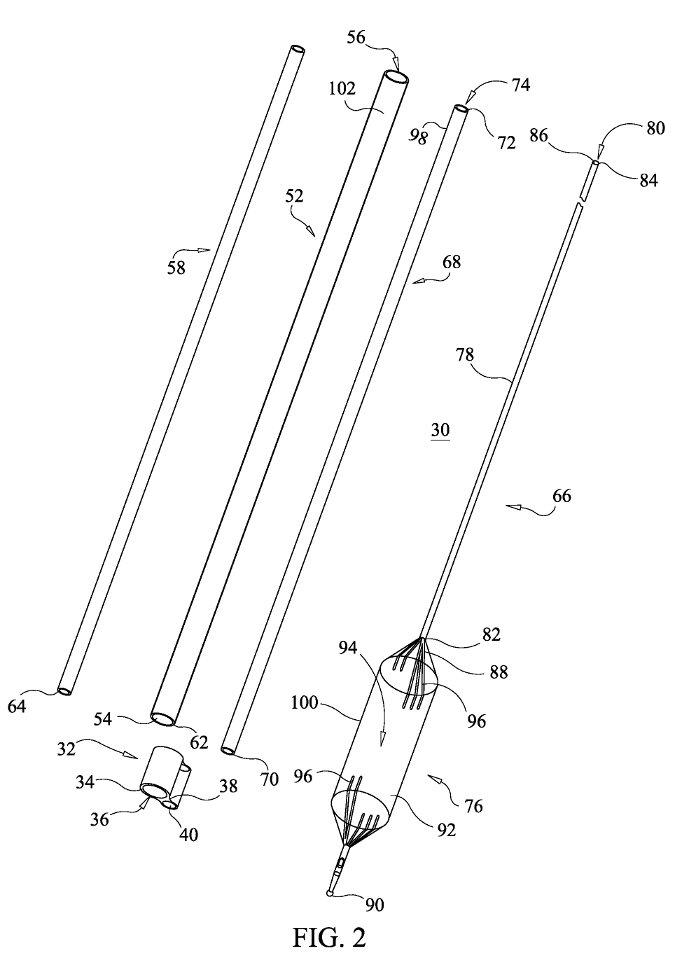

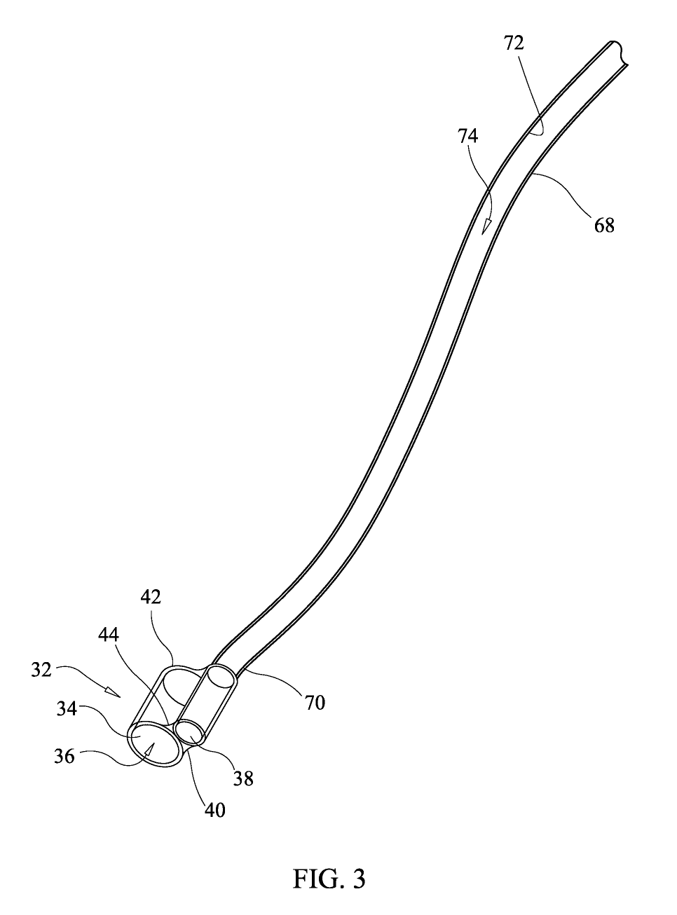

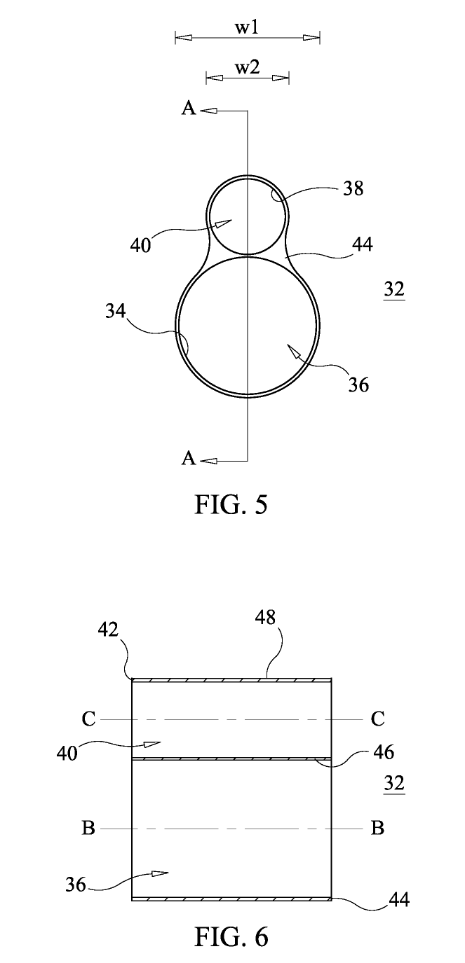

The following discussion includes a description of a surgical system that comprises an endoscope cap that includes a first channel for disposal of an endoscope and a second channel for disposal of devices in connection with the endoscope, such as, for example, a balloon catheter. Related kits and methods of employing the system in accordance with the principles of the present disclosure are also discussed. Alternate embodiments are also disclosed. Reference will now be made in detail to the exemplary embodiments of the present disclosure, which are illustrated in the accompanying figures. Turning now to FIGS. 1-6, there are illustrated components of a surgical system 30 and embodiments in accordance with the principles of the present disclosure.

System 30 comprises an endoscope cap, such as, for example, a sleeve 32 comprising an inner surface 34 defining a cylindrical first channel 36 that extends along a first longitudinal axis B and an inner surface 38 defining a cylindrical second channel 40 that extends along a second longitudinal axis C. Axis B is parallel to axis C. In some embodiments, channel 36 and/or channel 40 may have various cross section configurations, such as, for example, oval, oblong, triangular, rectangular, square, polygonal, irregular, uniform, non-uniform, variable, tubular and/or tapered. In some embodiments, axis C may be disposed at alternate orientations, relative to axis B, such as, for example, transverse, perpendicular and/or other angular orientations such as acute or obtuse, co-axial and/or may be offset or staggered.

Sleeve 32 comprises a flexible material, such as, for example, silicone or a flexible polymer, and is monolithically formed. In some embodiments, sleeve 32 comprises a single material. In some embodiments, sleeve 32 comprises a combination of materials. In some embodiments, sleeve 32 comprises low durometer silicone or a shape memory polymer. In some embodiments, sleeve 32 has a durometer of 10 Shore A to 60 Shore A. In some embodiments, sleeve 32 has a durometer of 20 Shore A to 30 Shore A. In some embodiments, sleeve 32 has a durometer of 25 Shore A. In some embodiments, sleeve 32 comprises an elastic material.

Channel 36 is spaced apart from channel 40 by a wall 46 that extends parallel to axes B, C. Channel 36 has a length defined by the distance between a first end surface 42 and a second end surface 44. Channel 40 has a length that is also defined by the distance between surface 42 and surface 44, the length of channel 36 being equivalent to that of channel 40. Channel 36 has a uniform width w1 along the entire length of channel 36. Channel 40 has a uniform width w2 along the entire length of channel 40. Width w1 is greater than width w2. In some embodiments, width w2 is about 4.0 mm to about 4.2 mm. It is envisioned that channel 40 may be variously configured with various widths or diameters to receive various sized tubes, for example, width w2 may be about 3.0 mm to about 5.0 mm or about 4.0 mm to about 5.0 mm. In some embodiments, the length of channels 36, 40 is between about 0.5 inches and about 3.0 inches.

An outer surface 48 of sleeve 32 is smooth so as to avoid injuring tissue and other portions of a patient's anatomy as sleeve 32 is inserted into an internal cavity of the patient, such as, for example, the patient's esophagus. In some embodiments, surface 48 may have various surface configurations to enhance fixation, such as, for example, rough, arcuate, undulating, porous, semi-porous, dimpled and/or textured, according to the requirements of a particular application.

A first surgical instrument, such as, for example, an endoscope 50 comprises a cylindrical shaft 52 disposed in channel 36. In some embodiments, sleeve 32 is configured to be flexible such that sleeve 32 can be stretched to fit over shaft 52. Shaft 52 includes an inner surface 54 defining a cylindrical first passageway 56. Endoscope 50 includes an optical probe 58 configured for illumination and/or imaging an internal cavity of a patient, such as, for example, the patient's esophagus. That is, in some embodiments, probe 58 is connected to a light source to provide light to illuminate an internal cavity of a patient's anatomy, such as, for example, the patient's esophagus. In some embodiments, probe 58 includes components configured for imaging of an internal cavity of a patient's anatomy, such as, for example, the patient's esophagus, using optical coherence tomography (OCT) techniques. Probe 58 is slidably disposed in passageway 56. Shaft 52 has a width that is less than width w1 such that an outer surface 60 of shaft 52 forms a friction fit with surface 34 when shaft 52 is disposed within channel 36 to engage shaft 52 with sleeve 32. In some embodiments, at least one of shaft 52 and probe 58 comprise a flexible material. In some embodiments, at least one of shaft 52 and probe 58 comprise a rigid material. In some embodiments, surface 60 includes surface configurations to enhance fixation with surface 34, such as, for example, rough, arcuate, undulating, porous, semi-porous, dimpled and/or textured. In some embodiments, shaft 52 and/or passageway 56 may have various cross section configurations, such as, for example, oval, oblong, triangular, rectangular, square, polygonal, irregular, uniform, non-uniform, variable, tubular and/or tapered.

Shaft 52 is disposed in channel 36 such that a first end surface 62 of shaft 52 is flush with surface 44. In some embodiments, shaft 52 is disposed in channel 36 such that surface 62 extends beyond surface 44. In some embodiments, shaft 52 is disposed in channel 36 such that surface 62 is positioned within channel 36. Probe 58 is positioned through passageway 56 such that a first end 64 of probe 58 protrudes through channel 36. In some embodiments, probe 58 is positioned through passageway 56 such that end 64 is flush with surface 62. In some embodiments, probe 58 is positioned through passageway 56 such that end 64 is positioned within passageway 56. In some embodiments, shaft 52 can be variously connected with sleeve 32, such as, for example, monolithic, integral connection, frictional engagement, threaded engagement, mutual grooves, screws, adhesive, nails, barbs and/or raised element.

A second surgical instrument, such as, for example, a balloon catheter 66 comprises a cylindrical utility tube 68 disposed in channel 40. Tube 68 is positioned within channel 40 such that a first end surface 70 of tube 68 is positioned adjacent surface 42. That is, surface 70 is positioned within channel 40 and is axially spaced apart from surface 44. In some embodiments, sleeve 32 comprises a flexible material such that surface 38 defines a radially flexible wall configured to stretch over tube 68. Tube 68 comprises an inner surface 72 defining a cylindrical second passageway 74. In some embodiments, tube 68 and/or passageway 56 may have various cross section configurations, such as, for example, oval, oblong, triangular, rectangular, square, polygonal, irregular, uniform, non-uniform, variable, tubular and/or tapered. In some embodiments, tube 68 is positioned within channel 40 such that surface 70 is flush with surface 44. In some embodiments, at least a portion of tube 68 is transparent to allow an inflatable balloon 76 and/or a cylindrical conduit 78 to be viewed through tube 68.

Conduit 78 is slidably disposed within passageway 74 and extends between a first end 80 and a second end 82. Conduit 78 includes an inner surface 84 defining a cylindrical lumen 86 configured for delivery of an inflation material into balloon 76, such as, for example, water, saline, a contrast solution or compressed air, to move balloon from a deflated or unexpanded orientation to an inflated or expanded orientation, as will be described. Conduit 78 comprises a compliant material, such as, for example, polyurethane, pellethane, polyethylene, silicone, cronoprene or non-compliant material such as nylon. In some embodiments, at least a portion of conduit 78 is transparent to allow inflation material to be viewed through conduit 78. In some embodiments, conduit 78 and/or lumen 86 may have various cross section configurations, such as, for example, circular, cylindrical, oval, oblong, triangular, rectangular, square, polygonal, irregular, uniform, non-uniform, variable, tubular and/or tapered.

Balloon 76 extends from a first end 88 and a second end 90. End 88 engages end 82. Balloon 76 and conduit 78 are integrally or monolithically formed. In some embodiments, balloon 76 is attached to conduit 78 by adhesive bonding, thermal bonding, laser bonding, or RF bonding. Balloon 76 includes an inner surface 92 defining an internal chamber 94 that is in communication with lumen 86 such that inflation material may be introduced through lumen 86 and into chamber 94 to move balloon 76 from a deflated or unexpanded configuration to an inflated or expanded configuration. That is, lumen 86 is continuous with chamber 94. Balloon 76 comprises a compliant material, such as, for example, polyurethane, pellethane, polyethylene, silicone, cronoprene or non-compliant material such as nylon. In some embodiments, balloon 76 comprises a material that is more complaint that the material from which conduit 78 is formed. In some embodiments, conduit 78 comprises a material configured to avoid expansion within passageway 74 as balloon 76 moves from the unexpanded orientation to the expanded orientation. In some embodiments, conduit 78 comprises a material that allows conduit 78 to expand such that an outer surface of conduit 78 engages surface 72 as balloon 76 moves from the unexpanded orientation to the expanded orientation in order to fix conduit 78 relative to tube 68. In some embodiments, at least a portion of balloon 76 is transparent to allow inflation material to be viewed through balloon 76 and/or to allow viewing of the tissue that defines the internal cavity of the patient. For example, proving a transparent balloon 76, may allow the internal cavity to be illuminated or imaged without balloon 76 interfering with such illumination or imaging.

It is envisioned that the shapes and sizes of balloon 76 when in the expanded configuration can be selected to provide a desired result during a procedure. For example, balloon 76 may include shapes such as spheres, cylinders, multi-lobed shapes, etc. and have different dimensions to make balloon 76 narrower or wider in a longitudinal direction, or extend further in a radial direction, etc. In some embodiments, balloon 76 includes one or a plurality of radiopaque markers 96. In some embodiments, balloon 76 includes at least one marker 96 at end 88 and at least one marker 96 at end 90. In some embodiments, the width of balloon 76 when balloon 76 is in the expanded orientation is about 10% to about 30% greater than width w2. In some embodiments, the width of balloon 76 when balloon 76 is in the expanded orientation is about 15% to about 20% greater than width w2.

In assembly, operation and use, system 30 is employed with a surgical procedure, such as, endoscopy. In some embodiments, system 30 is employed with an endoscopic procedure that utilizes OCT techniques. It is contemplated that one or all of the components of system 30 can be delivered or implanted as a pre-assembled device or can be assembled in situ. System 30 may be completely or partially revised, removed or replaced. It is envisioned that system 30 may also be used in connection with illuminating and/or imaging other portions of the patient's anatomy, such as, for example, other portions of the patient's gastrointestinal tract, portions of the patient's respiratory tract, the patient's ear, urinary tract, reproductive system, etc.

In use, a medical practitioner obtains access to an internal cavity of a patient's anatomy the medical practitioner desires to illuminate and/or image using an OCT technique, for example. Access to the portion of the patient's anatomy the medical practitioner desires to illuminate and/or image may be obtained in any appropriate manner. End 70 is inserted into channel 38 in the direction shown by arrow E. Selective positioning of end 70 within channel 38 may be provided by translating tube 68 within channel 38 in the direction shown by arrow E or the direction shown by arrow EE. In embodiments where sleeve 32 comprise a flexible material, sleeve 32 may be stretched so that sleeve 32 fits over tube 68, with end 70 positioned in channel 38 in the manner described above. Sleeve 32 therefore may also have to be stretched to accommodate selective positioning of end 70 within channel 38.

End 90 is inserted into passageway 74 at a second end 98 of tube 68 in the direction shown by arrow E. Balloon 76 is then translated within passageway 74 in the direction shown by arrow E until balloon 76 is positioned distal to sleeve 32, as shown in FIG. 1. When balloon 76 is positioned distal to sleeve 32, conduit 78 is positioned with passageway 74 such that end 80 is positioned proximal of tube 68, as also shown in FIG. 1. It is envisioned that catheter 68 may engage sleeve 32 either before or after sleeve 32 has been inserted into the internal cavity the medical practitioner desires to illuminate and/or image. In embodiments that include markers 96, the position of balloon 76 relative to the internal cavity the medical practitioner desires to illuminate and/or image may be determined by determining the position of markers 96. Balloon 76 hence may be selectively positioned within the internal cavity the medical practitioner desires to illuminate and/or image using markers 96.

Conduit 78 is connected to an inflation source, such as, for example, an inflation bulb, syringe or pump, the inflation source comprising an inflation material, such as, for example, the materials discussed above. Conduit 78 is connected to the inflation source such that the inflation material is delivered from the inflation source into lumen 86. The inflation material travels through lumen 86 in the direction shown by arrow E until the inflation material empties into chamber 94. As the inflation material fills chamber 94, balloon 76 moves from the unexpanded orientation to the expanded orientation. As balloon 76 moves from the unexpanded orientation to the expanded orientation, an outer surface 100 of balloon engages tissue that defines the internal cavity the medical practitioner desires to illuminate and/or image to fix sleeve 32 relative to the internal cavity. In some embodiments, catheter 66 is connected to a pressure gauge to allow the medical practitioner to monitor the pressure inside lumen 86 and/or chamber 94. The amount of inflation fluid introduced into chamber 94 from the inflation source may be adjusted if the pressure within lumen 86 and/or chamber 94 is too high or too low. It is envisioned the amount of inflation fluid introduced into chamber 94 from the inflation source may be adjusted by limiting the amount of inflation material that enters chamber 94 or withdrawing inflation material within chamber 94 such that the inflation material moves through lumen 86 in the direction shown by arrow EE and back into the inflation source.

End 70 is inserted into channel 38 in the direction shown by arrow E. Selective positioning of end 70 within channel 38 may be provided by translating tube 68 within channel 38 in the direction shown by arrow E or the direction shown by arrow EE. In embodiments where sleeve 32 comprise a flexible material, sleeve 32 may be stretched so that sleeve 32 fits over tube 68, with end 70 positioned in channel 38 in the manner described above. Sleeve 32 therefore may also have to be stretched to accommodate selective positioning of end 70 within channel 38.

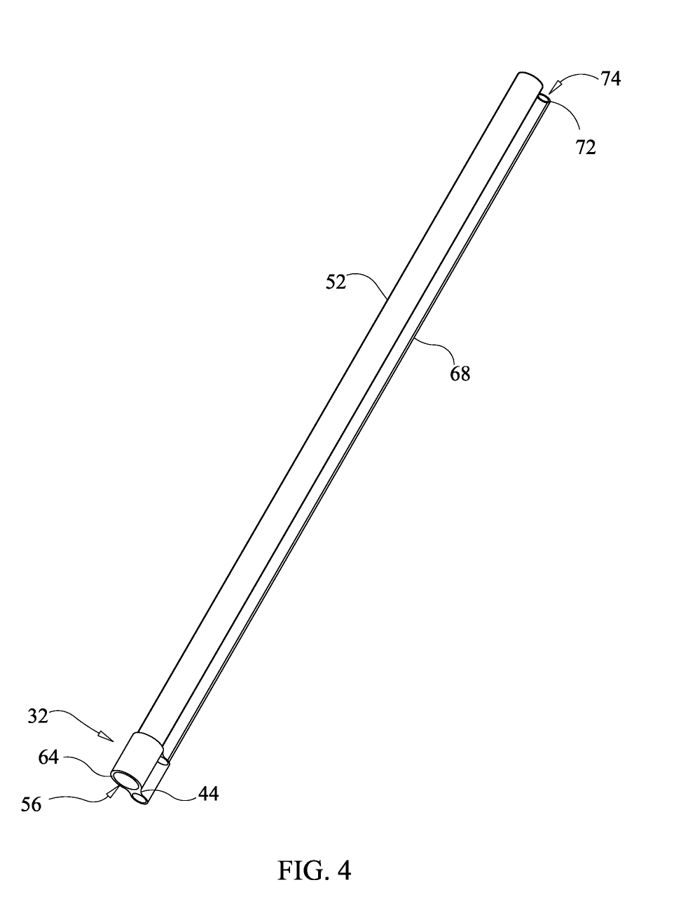

End 62 is inserted into channel 36 in the direction shown by arrow E. Shaft 52 is translated in the direction shown by arrow E within channel 36 until surface 62 is flush with surface 44, as shown in FIG. 4. End 64 is then inserted into passageway 56 at a second end 102 of shaft 52 in the direction shown by arrow E. Probe 58 is translated within passageway 56 in the direction shown by arrow E until end 64 is positioned distal to sleeve 32, as shown in FIG. 1. It is envisioned that probe 58 may be selectively positioned relative to sleeve 32 such that end 64 is flush with surface 44 or end 64 is positioned within passageway 56, as discussed above. Once probe 58 is positioned in sleeve 32 according to the preference of the medical practitioner, a second end 104 of probe 58 may be connected to an instrument, such as, for example a light source or other instrument used in connection with OCT techniques to illuminate and/or image of an internal cavity of a patient's anatomy. Illumination of at least a portion of the internal cavity and/or imaging of at least a portion of the internal cavity then may be accomplished using probe 58.

After illumination and/or imaging of the internal cavity is accomplished, probe 58 may be removed from shaft 52 by translating probe 58 in the direction shown by arrow EE. Shaft 52 may then be removed from sleeve 32 by translating shaft 52 in the direction shown by arrow EE. In embodiments where sleeve 32 comprises a flexible or elastic material, sleeve 32 may need to be stretched to release shaft 52 from channel 36.

Balloon 76 is moved from the expanded orientation to the unexpanded orientation by withdrawing the inflation fluid from chamber 94 through lumen 86 in the direction shown by arrow 86. After balloon 76 is in the unexpanded configuration, balloon 76 may be translated within passageway 74 in the direction shown by arrow EE until balloon is exits passageway 74 at end 98. Once balloon 76 is removed from passageway 74, tube 68 may be removed from channel 40 by translating tube 68 relative to sleeve in the direction show by arrow EE. In embodiments where sleeve 32 comprises a flexible or elastic material, sleeve 32 may need to be stretched to release tube 68 from channel 40. It is envisioned that tube 68 may be removed from channel 40 either before or after sleeve is removed from the patient's anatomy.

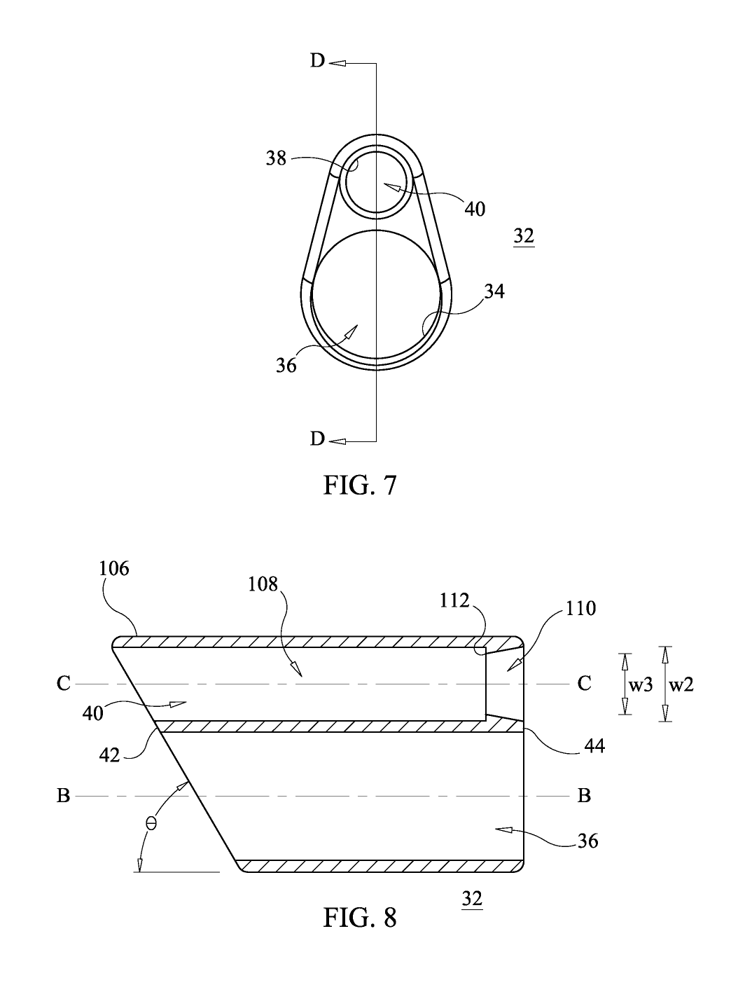

In one embodiment, shown in FIGS. 7 and 8, surface 42 extends at an angle θ relative to axes B, C and surface 44 extends perpendicular to axes B, C such that a first end 106 of sleeve 32 has a tapered configuration. In some embodiments, angle θ is an acute angle. In some embodiments, angle θ is an angle between 40° and 80°. In some embodiments, angle θ is an angle between 45° and 75°. In some embodiments, angle θ is 60°. The angled configuration of surface 42 provides that maximum length of channel 36 is less than that of channel 40, as shown in FIG. 8. Having surface 42 extend at an angle θ relative to axes B, C provides sleeve 32 with a wedge-shape. As will be appreciated by one of ordinary skill in the art, angled surface 42 allows shaft 52 and/or tube 68 to be inserted into channels 36, 40 more easily and serves as a lead-in for such components.

As shown in FIG. 8, a first portion 108 of passageway 40 has a cylindrical configuration and a second portion 110 of passageway 40 has a chamfered configuration. An interface between portions 108, 110 defines a circumferential flange 112 that extends perpendicular to axes B, C. Channel 40 has a maximum width w3 between flange 112 that is less than width w2. In some embodiments, conduit 78 has a width that is less than width w2 but greater than width w3 and balloon 76, when in its unexpanded orientation, has a width that is less than width w3 such that end 82 is prevented from being inserted into portion 110. That is, flange 112 acts as a stop member to prevent end 82 from entering portion 110. In some embodiments, end 88 has a conical configuration when balloon 76 is in the expanded orientation that matches the chamfered configuration of portion 110 in a manner that allows portion 110 to orient balloon 76 in a direction that is parallel with axes B, C.

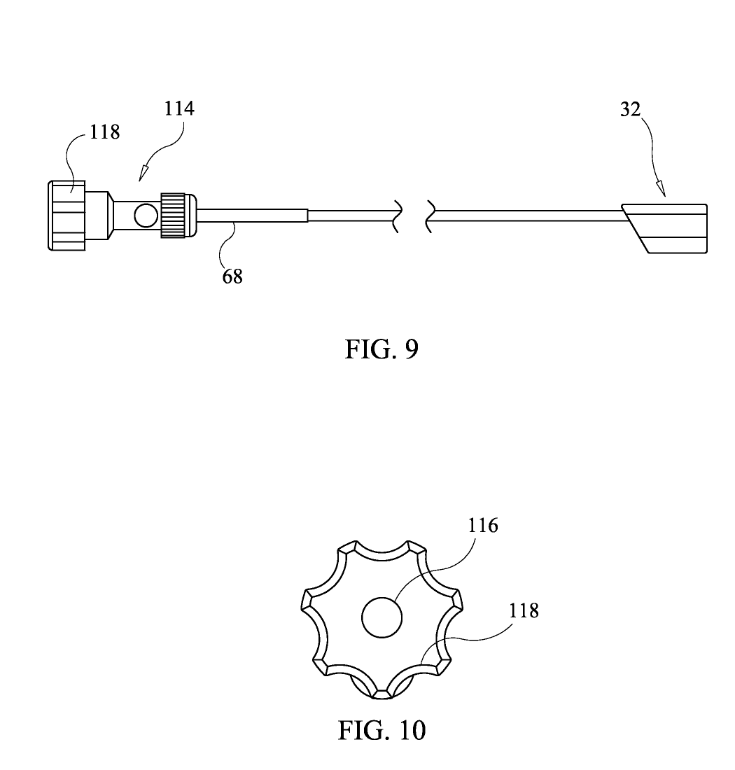

In one embodiment, shown in FIGS. 9 and 10, system 30 includes an adapter 114 comprising an opening having tube 68 positioned therein. Adapter 114 includes a gasket 116 in a body of adapter 114 that can be tightened about tube 68 by turning a knob 118. Once gasket 116 is tightened, fluid from an inflation source, for example, will not leak passed tube 68. In one embodiment, adaptor 114 is a Touhy-Borst adapter.

In one embodiment, system 30 includes magnetic tracking capability such that the components of system 30, including, for example, endoscope 50, catheter 66 and/or sleeve 32 can be tracked in 3D. Radiomarkers may be included for identification under x-ray, fluoroscopy, CT or other imaging techniques. It is envisioned that the use of microsurgical and image guided technologies may be employed to access, view and repair spinal deterioration or damage, with the aid of system 30.

One of ordinary skill in the art should appreciate that the configuration of shaft 52 is such that various types of surgical instruments can be disposed within passageway 56 for use with many different diagnostic procedures. For example, it is envisioned that a heater probe or an ultrasonic probe may be positioned within passageway 56 in lieu of optical probe 58, where a medical practitioner requires use of heater probe or an ultrasonic probe, rather than an optical probe. It is further envisioned that system 30 may include such various types of surgical instruments such that the surgical instruments, including probe 58 can be interchangeably inserted into passageway 56, according to the use required by the medical practitioner. It is envisioned that the kit described above may include the various types of surgical instruments discussed in this paragraph. It is further envisioned that the method described above may also include using of the various types of surgical instruments discussed in this paragraph, as an alternative to, or in addition to, the steps discussed above.

Since the components of system 30 will be inserted into an internal cavity of a patient, such as, for example, the patient' esophagus, it is advantageous to provide components that are biologically acceptable materials suitable for medical applications. The biologically acceptable materials suitable for medical applications for system 30 can be fabricated from and include metals, synthetic polymers, ceramics and bone material and/or their composites, depending on the particular application and/or preference of a medical practitioner. For example, the components of system 30, individually or collectively, can be fabricated from materials such as stainless steel alloys, commercially pure titanium, titanium alloys, Grade 5 titanium, super-elastic titanium alloys, cobalt-chrome alloys, stainless steel alloys, superelastic metallic alloys (e.g., Nitinol, super elasto-plastic metals, such as GUM METAL® manufactured by Toyota Material Incorporated of Japan), ceramics and composites thereof such as calcium phosphate (e.g., SKELITE™ manufactured by Biologix Inc.), thermoplastics such as polyaryletherketone (PAEK) including polyetheretherketone (PEEK), polyetherketoneketone (PEKK) and polyetherketone (PEK), carbon-PEEK composites, PEEK-BaSO₄ polymeric rubbers, polyethylene terephthalate (PET), PVC, fabric, silicone, polyurethane, silicone-polyurethane copolymers, polymeric rubbers, polyolefin rubbers, hydrogels, semi-rigid and rigid materials, elastomers, rubbers, thermoplastic elastomers, thermoset elastomers, elastomeric composites, rigid polymers including polyphenylene, polyamide, polyimide, polyetherimide, polyethylene, epoxy, and their combinations.

Various components of system 30 may have material composites, including the above materials, to achieve various desired characteristics such as strength, rigidity, elasticity, compliance, mechanical performance and durability. The components of system 30, individually or collectively, may also be fabricated from a heterogeneous material such as a combination of two or more of the above-described materials. The components of system 30 may be monolithically formed, integrally connected or include fastening elements and/or instruments, as described herein.

While the preferred embodiments of the systems, kits and methods have been described in reference to the environment in which they were developed, they are merely illustrative of the principles of the present disclosure. Modification or combinations of the above-described systems, kits and methods and variations of aspects thereof that are obvious to those of skill in the art are intended to be within the scope of the present disclosure.

What is claimed is:

1. A surgical system, comprising:
a sleeve comprising an inner surface defining a first channel that extends along a first longitudinal axis and an inner surface defining a second channel that extends along a second longitudinal axis, the second channel being spaced apart from the first channel;
a first surgical instrument configured for disposal in the first channel, the first surgical instrument comprising an optical probe configured for illumination and/or imaging; and
a second surgical instrument configured for disposal in the second channel, the second surgical instrument comprising an inflatable balloon.

2. A system according to claim 1, wherein the first surgical instrument is an endoscope and the second surgical instrument is a balloon catheter.

3. A system according to claim 1, wherein the first longitudinal axis extends parallel to the second longitudinal axis.

4. A system according to claim 1, wherein a width of the first channel is greater than that of the second channel.

5. A system according to claim 1, wherein a width of the second channel is about 4.0 mm to about 4.2 mm and a width of the balloon is about 25 mm when the balloon is in an expanded orientation.

6. A system according to claim 1, wherein a width of the balloon when the balloon is in an expanded orientation is about 10% to about 30% greater than a width of the second channel.

7. A system according to claim 1, wherein the first channel has a width that is only slightly greater than a width of an outer surface of the first surgical instrument such that the sleeve and the first surgical instrument form a friction fit to provisionally fix the first surgical instrument relative to the sleeve.

8. A system according to claim 1, wherein the second surgical instrument is slidably disposed in the second channel.

9. A system according to claim 1, wherein the sleeve comprises low durometer silicone.

10. A system according to claim 1, wherein the sleeve comprises a shape memory polymer.

11. A system according to claim 1, wherein a length of the first channel is equal to that of the second channel.

12. A system according to claim 1, wherein a maximum length of the first channel is less than that of the second channel.

13. A system according to claim 1, wherein the second channel has a uniform width.

14. A system according to claim 1, wherein a first end of the second channel has a chamfered configuration.

15. A system according to claim 1, wherein the inflatable balloon comprises radiopaque markers.

16. A method for catheter deployment, comprising:
providing the system of claim 1;
inserting a utility tube of the second surgical instrument into the second channel, the utility tube comprising an inner surface defining a first passageway;
inserting the balloon through the first passageway with the balloon in an unexpanded orientation;
selectively positioning the balloon in an internal cavity of a patient;
moving the balloon from the unexpanded orientation to an expanded orientation such that the balloon engages tissue that defines the internal cavity to fix the balloon relative to the internal cavity;
inserting a shaft of the first surgical instrument into the first channel, the shaft comprising an inner surface defining a second passageway;
inserting the optical probe through the second passageway and into the internal cavity; and
illuminating and/or imaging the internal cavity using the optical probe.

17. A method for catheter deployment, wherein the internal cavity is the patient's esophagus.

18. A kit for an endoscope, comprising:
the system of claim 1;
one or more additional sleeves; and
one or more additional second surgical instruments, the inflatable balloons of each of the second surgical instruments having a different configuration when in an expanded orientation.

19. A surgical system, comprising:
a sleeve comprising an inner surface defining a first channel that extends along a first longitudinal axis and an inner surface defining a second channel that extends along a second longitudinal axis, the second longitudinal axis being parallel to the first longitudinal axis, the first channel having a width that is greater than that of the second channel, the widths of the first and second channels each being uniform, the second channel being spaced apart from the first channel, the sleeve comprising a shape memory polymer or low durometer silicone;
an endoscope comprising a shaft configured for disposal in the first channel, the shaft including an inner surface defining a first passageway having an optical probe configured for illumination and/or imaging slidably disposed therein; and
a balloon catheter comprising a utility tube configured for disposal in the second channel, the utility tube comprising an inner surface defining a second passageway configured for disposal of an inflatable balloon,
wherein the first and second channels extend between first and second end surfaces of the sleeve, the first and second end surfaces each extending perpendicular to the first and second longitudinal axes such that a length of the first channel is equal to that of the second channel.

20. A surgical system, comprising:
a sleeve comprising an inner surface defining a first channel that extends along a first longitudinal axis and an inner surface defining a second channel that extends along a second longitudinal axis, the second longitudinal axis being parallel to the first longitudinal axis, the first channel having a maximum width that is greater than that of the second channel, the second channel being spaced apart from the first channel, a first portion of the second channel having a cylindrical cross sectional configuration and a second portion of the second channel having a tapered configuration, the sleeve comprising a shape memory polymer or low durometer silicone;
an endoscope comprising a shaft configured for disposal in the first channel, the shaft including an inner surface defining a first passageway having an optical probe configured for illumination and/or imaging slidably disposed therein; and
a balloon catheter comprising a utility tube configured for disposal in the second channel, the utility tube comprising an inner surface defining a second passageway configured for disposal of an inflatable balloon,
wherein the first and second channels extend between first and second end surfaces of the sleeve, the first end surface extending at an acute angle relative to the first and second longitudinal axes and the second end surface extending perpendicular to the first and second longitudinal axes such that a length of the first channel is less than that of the second channel.
